(19) United States
(12) Patent Application Publication
(10) Pub. No.: US 2007/0031496 A1
(43) Pub. Date: Feb. 8, 2007

(54) Osmotic dosage forms comprising semipermeable membranes with polymer blends providing improved properties

(76) Inventors: David E. Edgren, Los Altos, CA (US); Shu Li, Union City, CA (US); Gurdish K. Bhatti, Fremont, CA (US); Patrick S.L. Wong, Burlingame, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/499,008

(22) Filed: Aug. 4, 2006

Related U.S. Application Data
(60) Provisional application No. 60/705,934, filed on Aug. 4, 2005.

(51) Int. Cl. A61K 9/24

(52) U.S. Cl. 424/473

(57) ABSTRACT

Disclosed are osmotic dosage forms including an osmotic core; a semi-permeable membrane that surrounds the osmotic core and comprises a blend of a cellulose acetate polymer and an acrylate copolymer; and an exit formed through the semi-permeable membrane. Also disclosed are methods of making and administering such dosage forms.

### CROSS REFERENCE TO RELATED U.S. APPLICATION INFO

The present application claims the benefit under 35 U.S.C. 119(e) of Provisional application 60/705,934 filed Aug. 4, 2005.

### FIELD OF THE INVENTION

The invention relates to osmotic dosage forms, particularly to osmotic dosage forms comprising a semi-permeable membranes that comprise a blend of a cellulose acetate polymer and an acrylate copolymer. These inventive osmotic dosage forms display surprising and unexpected performance in operation.

### BACKGROUND

Osmotic dosage forms in general utilize osmotic pressure to generate a driving force for imbibing fluid into a compartment formed, at least in part, by a semipermeable membrane that permits free diffusion of fluid but not drug. A significant advantage to osmotic systems is that operation is pH-independent and thus continues at the osmotically determined rate throughout an extended time period even as the dosage form transits the gastrointestinal tract and encounters differing microenvironments having significantly different pH values. Simple osmotic pumps require membranes that are sufficiently rigid to resist mechanical deformation by external peristaltic forces in the gastrointestinal tract to prevent environment controlled bellows type mechanical pumping. A review of such dosage forms is found in Santus and Baker, “Osmotic drug delivery: a review of the patent literature,” Journal of Controlled Release 35 (1995) 1-21, incorporated by reference herein. U.S. Pat. Nos. 3,845,770; 3,916,899; 3,995,631; 4,008,719; 4,111,202; 4,160,020; 4,327,725; 4,519,801; 4,578,075; 4,681,583; 5,019,397; and 5,156,850 disclose osmotic devices for the continuous dispensing of active agent.

Osmotic dosage forms in which a drug composition is delivered as a slurry, suspension or solution from a small exit orifice by the action of an expandable layer are disclosed in U.S. Pat. Nos. 4,931,285; 5,006,346; 5,024,842; 5,160,743; 5,190,765; 5,190,765; 5,252,338; 5,620,705; and 5,633,011 which are incorporated herein by reference. Typical devices include an expandable push layer and a drug layer surrounded by a semipermeable membrane. Such systems require rate-controlling membranes that provide good tensile properties while the system is pumping drug such that as the internal hydrogels swell within the dosage form under osmotic swelling forces, the membranes can resist deformation and resist tearing under forces of tension.

In certain circumstances, it is desirable to adjust the permeability of the semi-permeable membrane in osmotic dosage forms. This may be desirable when a lower water flux into the membrane is needed, for instance to provide a slower rate of drug release from the osmotic dosage form. Conventionally, water flux may be modulated by varying the coating thickness of the semi-permeable membrane. However, this may result in very thick coatings for achieving lower water flux, which very thick coatings adversely affect the cost and capacity of manufacturing process by increasing the amount of coating materials that are required to be applied, increasing the processing time to apply the coatings, and increasing the processing time required to dry residual processing solvents to acceptably low levels.

Accordingly, methods and compositions are in critical need that provide thinner, less permeable semipermeable membranes and that address the problems in the prior art noted above.

### SUMMARY OF THE INVENTION

In an embodiment, the invention relates to an osmotic dosage form comprising: an osmotic core; a semi-permeable membrane that surrounds the osmotic core and comprises a blend of a cellulose acetate polymer and an acrylate copolymer; and an exit formed through the semi-permeable membrane.

In another embodiment, the invention relates to a method comprising: providing an osmotic core; coating the osmotic core with a semi-permeable membrane that comprises a blend of a cellulose acetate polymer and an acrylate copolymer; forming an exit through the semi-permeable membrane; and administering the coated osmotic core to a patient.

### BRIEF DESCRIPTION OF THE DRAWINGS

FIG. 1 shows an osmotic dosage form according to the invention.

FIG. 2 shows another osmotic dosage form according to the invention.

FIG. 3 shows results from testing of semipermeable membranes.

FIG. 4 shows results from additional testing of semipermeable membranes.

FIG. 5 shows delivery patterns from osmotic dosage forms.

FIG. 6 shows the results from a drying study using osmotic dosage forms.

### DETAILED DESCRIPTION

Introduction

The inventors have unexpectedly discovered that the problems in the prior art can be addressed by the use of a blend of a cellulose acetate polymer and an acryl ate copolymer in the semi-permeable membranes of osmotic dosage forms.

The inventive blends provide for lower permeability, and also provide good mechanical properties. It is unexpected to successfully blend a cellulose acetate polymer with an acrylate copolymer for such polymers possess such globally different properties as to lead one of skill away from blending one with the other. The cellulose acetate polymer is a derivative of naturally occurring cellulose, the structural polymer of plant origin. The functional groups of the cellulose derivative of the present invention are acetyl groups. The cellulose acetate of the present invention is a hard material with a glass transition point of 185° C. and a molecular weight ranging from about 38,000 to 122,000. By contrast, the acrylate polymer of the blend is of completely synthetic origin with ethoyxl and methoxyl functional groups and without acetyl groups. The acrylate polymer is a very soft material with a glass transition point of about −11° C. The molecular weights of the two polymers are completely dissimilar with the molecular weight of the acrylate being about 800,000 grams per mole. It is common that polymer blends of dissimilar structure often lead to mixture blends that have poor properties. These two polymers of such widely different structure provide blends that surprisingly form blended membranes with useful permeability and mechanical properties. Moreover, the physical forms of the two polymers as supplied by the manufacturers are not supplied in forms conducive to and lead away from blending. The cellulose acetate is supplied by the manufacturer as a dry powder which is intended to be used as a dry powder by first dissolving in an organic solvent to form a coating solution and then used to produce a coating. By contrast, the acrylate copolymer is supplied by the manufacturer and is intended to be used as a dispersion in water without organic solvents to be directly applied from water to produce a coating.

The invention, and embodiments thereof, will now be described in more detail.

Definitions

All documents cited to herein are hereby incorporated by reference, for all purposes and in their entirety as if reproduced fully herein.

The present invention is best understood by reference to the following definitions, the drawings and exemplary disclosure provided herein.

“Acrylate copolymer” means a linear copolymer comprised of alkyl acrylic acid monomer units where at least two different alkyl acrylic acid monomer units are polymerized to comprise the copolymer. Acrylate copolymer are discussed further elsewhere herein.

“Administering” means providing a material, especially a drug, to a patient.

“Blend means a polymer composition comprising at least two polymers that are physically mixed together and function in concert differently than each polymer individually.

“Cellulose acetate polymer” means a linear polymer comprised of repeat units of anhydroglucose which hydroxyl groups of the anhydroglucose units on average are substituted with greater than zero and up to three acetyl groups per anhydroglucose unit. Cellulose acetate polymers are discussed further elsewhere herein.

“Coating” means providing a film over a substrate.

“Dosage form” means a material suitable for pharmaceutical administration to a patient.

“Exit” or “Exit port” means an opening, bore, or, channel which connects the interior of an osmotic dosage form to an exterior environment of use.

“Osmotic core” means a formed composition that comprises at least one osmotically active substance and at least one drug wherein the osmotic core is intended for use within an osmotic dosage form.

“Osmotic dosage form” means a dosage form that operates according to osmotic principles to deliver a unit dose of drug at a controlled rate for a controlled duration of time. Osmotic dosage forms, in general, utilize osmotic pressure to generate a driving force for imbibing fluid into a compartment formed, at least in part, by a semipermeable wall. An advantage to osmotic dosage forms is that their operation is pH-independent and, thus, continues at the osmotically determined rate throughout an extended time period even as the osmotic dosage form transits the gastrointestinal tract and encounters differing microenvironments having significantly different pH values. A review of such osmotic dosage forms is found in Santus and Baker, “Osmotic drug delivery: a review of the patent literature,38 Journal of Controlled Release, 35:1-21 (1995). Osmotic dosage forms are also described in detail in the following U.S. patents, each incorporated in their entirety herein: U.S. Pat. Nos. 3,845,770; 3,916,899; 3,995,631; 4,008,719; 4,111,202; 4,160,020; 4,327,725; 4,519,801; 4,578,075; 4,681,583; 5,019,397; and 5,156,850.

“Patient” means a person or animal that is the object of study and/or medical intervention.

“Semipermeable membrane” means a membrane that permits free diffusion of fluids such as water or other fluids in an external environment of use but not of a drug or an osmotic agent(s).

“Water flux” means a volumetric flow per unit time of water across a semipermeable membrane per unit area of semipermeable membrane.

Osmotic dosage forms according to the invention comprise semipermeable membranes that surround an osmotic core. Such structures are discussed further elsewhere herein. Materials useful for forming the semi-permeable membrane are essentially nonerodible and are substantially insoluble in biological fluids during the life of the dosage form.

The osmotic dosage forms of the present invention comprise semipermeable membranes that comprise a blend of a cellulose acetate polymer and of an acrylate copolymer. The blended semipermeable membrane provides transport and mechanical properties appropriate and needed for the proper functioning of an osmotic dosage form. Permeability of the semipermeable membrane blend can be targeted to a level such that the osmotic dosage form delivers the drug at the appropriate rate and for the appropriate duration for an osmotic dosage form administered to a patient in need while also providing the mechanical properties appropriate for the delivery device. The ratio of the two polymers can be selected to provide the appropriate transport and mechanical properties. The percentage of acrylate copolymer of the blend is adjusted upwards to provide a reduction in permeability. In an embodiment, the blend comprises the cellulose acetate polymer in an amount equal to or greater than about 50 wt % based on a total dry weight of the blend, preferably the blend comprises the cellulose acetate polymer in an amount equal to or greater than about 60 wt % based on a total dry weight of the blend; more preferably the blend comprises the cellulose acetate polymer in an amount equal to or greater than about 70 wt %, based on a total dry weight of the blend. A 50:50 blend of cellulose acetate and acrylate copolymer is approximately one third the permeability of the cellulose acetate in the absence of the acrylate fraction. In an embodiment, the blend comprises acrylate copolymer in an amount ranging from about 0.01 wt % to about 50 wt %, based on a total dry weight of the blend; preferably the blend comprises acrylate copolymer in an amount ranging from about 10 wt % to about about 30 wt %, based on a total dry weight of the blend; more preferably the blend comprises acrylate copolymer in an amount ranging from about 15 wt % to about 25 wt %, based on a total dry weight of the blend.

The semi-permeable membrane may also comprise an optional flux-regulating agent. The flux-regulating agent is a compound added to assist in regulating the fluid permeability or flux through the semi-permeable membrane. The flux-regulating agent can be a flux enhancing agent. The agent can be pre-selected to increase the liquid flux across the membrane blend. Agents that produce a marked increase in permeability to fluids such as water are often essentially hydrophilic. The amount of flux regulator in semi-permeable membrane when incorporated therein generally ranges from about 0.01 wt % to about 25 wt % by weight, preferably about 5 wt % to about 20 wt %, more preferably about 10 wt % to about 15 wt %, based on the total dry weight of the blend.

The flux regulator agents in one embodiment that increase flux include, for example, polyhydric alcohols, polyalkylene glycols, polyalkylenediols, polyesters of alkylene glycols, and the like. Typical flux enhancers include polyethylene glycol 300, 400, 600, 1500, 4000, 6000, poly(ethylene glycol-co-propylene glycol), ethylene oxide: propylene oxide:ethylene oxide triblock copolymers, and the like; low molecular weight gylcols such as polypropylene glycol, polybutylene glycol and polyamylene glycol: the polyalkylenediols such as poly(1,3-propanediol), poly(1,4-butanediol), poly(1,6-hexanediol), and the like; aliphatic diols such as 1,3-butylene glycol, 1,4-pentamethylene glycol, 1,4-hexamethylene glycol, and the like; alkylene triols such as glycerine, 1,2,3-butanetriol, 1,2,4-hexanetriol, 1,3,6-hexanetriol and the like; esters such as ethylene glycol dipropionate, ethylene glycol butyrate, butylene glycol dipropionate, glycerol acetate esters, monosaccharides such a sorbitol, disaccharides such as lactose, combinations of various flux regulators, and the like.

Other materials that can be used to form the semi-permeable membrane for imparting flexibility and elongation properties to the wall, for making the wall less-to-nonbrittle and to render tear strength, include, for example, phthalate plasticizers such as dibenzyl phthalate, dihexyl phthalate, butyl octyl phthalate, straight chain phthalates of six to eleven carbons, di-isononyl phthalate, di-isodecyl phthalate, and the like. The plasticizers include nonphthalates such as triacetin, dioctyl azelate, epoxidized tallate, tri-isoctyl trimellitate, tri-isononyl trimellitate, sucrose acetate isobutyrate, epoxidized soybean oil, lecithin, and the like. The amount of plasticizer in a semi-permeable membrane when incorporated therein is about 0.01% to about 20% weight, or higher.

The inventive semi-permeable membranes may be coated onto the osmotic cores using techniques known in the art and/or disclosed elsewhere herein.

### Dosage Forms

Osmotic dosage forms and methods of treatment using the osmotic dosage forms will now be described. It will be appreciated that the osmotic dosage forms described below are merely exemplary.

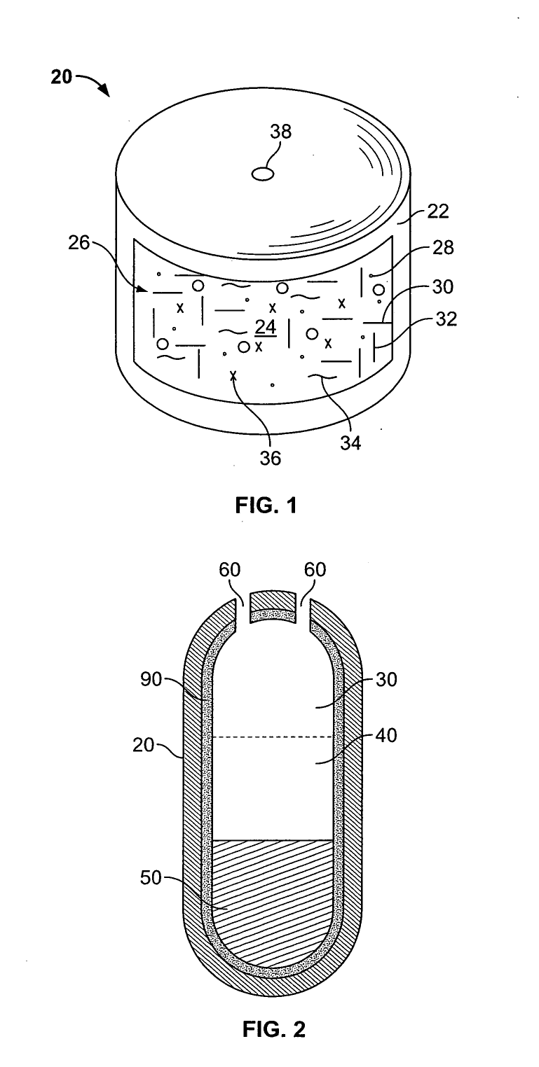

An exemplary osmotic dosage form, referred to in the art as an elementary osmotic pump dosage form, is shown in FIG. 1. Dosage form 20, shown in a cutaway view, is also referred to as an elementary osmotic pump, and is comprised of a semi-permeable wall 22 that surrounds and encloses an internal compartment 24. The internal compartment contains a single component layer referred to herein as a drug layer 26, comprising an inventive substance 28 in an admixture with selected excipients. The excipients are adapted to provide an osmotic activity gradient for attracting fluid from an external environment through wall 22 and for forming a deliverable complex formulation upon imbibition of fluid. The excipients may include a suitable suspending agent, also referred to herein as drug carrier 30, a binder 32, a lubricant 34, and an osmotically active agent referred to as an osmagent 36. Exemplary materials useful for these components can be found disclosed throughout the present application.

Semi-permeable membrane 22 of the osmotic dosage form is permeable to the passage of an external fluid, such as water and biological fluids, but is substantially impermeable to the passage of components in the internal compartment.

In operation, the osmotic gradient across semi-permeable membrane 22 due to the presence of osmotically-active agents causes fluid of the gastrointestinal tract to be imbibed through the wall, swelling of the drug layer, and formation of a deliverable complex formulation (e.g., a solution, suspension, slurry or other flowable composition) within the internal compartment. The deliverable inventive substance formulation is released through an exit port 38 as fluid continues to enter the internal compartment. Even as drug formulation is released from the dosage form, fluid continues to be drawn into the internal compartment, thereby driving continued release. In this manner, the inventive substance is released in a sustained and continuous manner over an extended time period.

FIG. 2 illustrates certain inventive embodiments of sustained release dosage forms. Dosage forms of this type are described in detail in U.S. Pat. Nos.: 4,612,008; 5,082,668; and 5,091,190; and are further described below

FIG. 2 shows an embodiment of one type of sustained release dosage form, namely the osmotic sustained release dosage form. First drug layer 30 comprises osmotically active components, and a lower amount of active agent than in second drug layer 40. The osmotically active component(s) in the first component drug layer comprises an osmagent such as salt and one or more osmopolymer(s) having relatively small molecular weights which exhibit swelling as fluid is imbibed such that release of these osmopolymers through exit port 60 occurs similar to that of drug layer 40. Additional excipients such as binders, lubricants, antioxidants and colorants may also be included in first drug layer 30.

Second drug layer 40 comprises active agent in an admixture with selected excipients adapted to provide an osmotic activity gradient for driving fluid from an external environment through semi-permeable membrane 20 and for forming a deliverable drug formulation upon imbibition of fluid. The excipients may include a suitable suspending agent, also referred to herein as a drug carrier, but no osmotically active agent, “osmagent,” such as salt, sodium chloride. It has been discovered that the omission of salt from this second drug layer, which contains a higher proportion of the overall drug in the dosage form, in combination with the salt in the first drug layer, provides an improved ascending rate of release creating a longer duration of ascending rate.

Drug layer 40 has a higher concentration of the drug than does drug layer 30. The ratio of the concentration of drug in the first drug layer 30 to the concentration of drug in the second drug layer 40 is maintained at less than 1 and preferably less than or equal to about 0.43 to provide the desired substantially ascending rate of release.

Drug layer 40 may also comprise other excipients such as lubricants, binders, etc.

Drug layer 40, as with drug layer 30, further comprises a hydrophilic polymer carrier. Representative examples of these polymers are poly(alkylene oxide) of 100,000 to 750,000 number-average molecular weight, including poly(ethylene oxide), poly(methylene oxide), poly(butylene oxide) and poly(hexylene oxide); and a poly(carboxymethylcellulose) of 40,000 to 400,000 number-average molecular weight, represented by poly(alkali carboxymethylcellulose), poly(sodium carboxymethylcellulose), poly(potassium carboxymethylcellulose) and poly(lithium carboxymethylcellulose). Drug layer 40 can further comprise a hydroxypropylalkylcellulose of 9,200 to 125,000 number-average molecular weight for enhancing the delivery properties of the dosage form as represented by hydroxypropylethylcellulose, hydroxypropylmethylcellulose, hydroxypropylbutylcellulose and hydroxypropylpentylcellulose; and a poly(vinylpyrrolidone) of 7,000 to 75,000 number-average molecular weight for enhancing the flow properties of the dosage form. Preferred among these polymers are the poly(ethylene oxide) of 100,000-300,000 number average molecular weight. Carriers that erode in the gastric environment, i.e., bioerodible carriers, are especially preferred.

Other carriers that may be incorporated into drug layer 40, and/or drug layer 30, include carbohydrates that exhibit sufficient osmotic activity to be used alone or with other osmagents. Such carbohydrates comprise monosaccharides, disaccharides and polysaccharides. Representative examples include maltodextrins (i.e., glucose polymers produced by the hydrolysis of corn starch) and the sugars comprising lactose, glucose, raffinose, sucrose, mannitol, sorbitol, and the like. Preferred maltodextrins are those having a dextrose equivalence (DE) of 20 or less, preferably with a DE ranging from about 4 to about 20, and often 9-20. Maltodextrin having a DE of 9-12 has been found to be useful.

Drug layer 40 and drug layer 30 typically will be a substantially dry, <5% water by weight, composition formed by compression of the carrier, the drug, and other excipients as one layer.

Drug layer 40 may be formed from particles by comminution that produces the size of the drug and the size of the accompanying polymer used in the fabrication of the drug layer, typically as a core containing the compound, according to the mode and the manner of the invention. The means for producing particles include granulation, spray drying, sieving, lyophilization, crushing, grinding, jet milling, micronizing and chopping to produce the intended micron particle size. The process can be performed by size reduction equipment, such as a micropulverizer mill, a fluid energy grinding mill, a grinding mill, a roller mill, a hammer mill, an attrition mill, a chaser mill, a ball mill, a vibrating ball mill, an impact pulverizer mill, a centrifugal pulverizer, a coarse crusher and a fine crusher. The size of the particle can be ascertained by screening, including a grizzly screen, a flat screen, a vibrating screen, a revolving screen, a shaking screen, an oscillating screen and a reciprocating screen. The processes and equipment for preparing drug and carrier particles are disclosed in Pharmaceutical Sciences, Remington, 17th Ed., pp. 1585-1594 (1985); Chemical Engineers Handbook, Perry, 6th Ed., pp. 21-13 to 21-19 (1984); Journal of Pharmaceutical Sciences, Parrot, Vol. 61, No. 6, pp. 813-829 (1974); and Chemical Engineer, Hixon, pp. 94-103 (1990).

First drug layer 30 comprises active agent in an admixture with selected excipients adapted to provide an osmotic activity gradient for driving fluid from an external environment through semi-permeable membrane 20 and for forming a deliverable drug formulation upon imbibition of fluid. The excipients may include a suitable suspending agent, also referred to herein as a drug carrier, and an osmotically active agent, i.e., an “osmagent,” such as salt. Other excipients such as lubricants, binders, etc. may also be included.

The osmotically active component in the first drug layer typically comprises an osmagent and one or more osmopolymer(s) having relatively small molecular weights which exhibit swelling as fluid is imbibed such that release of these osmopolymers through exit port 60 occurs similar to that of drug layer 40.

The ratio of drug concentration between the first drug layer and the second drug layer alters the release rate profile. Release rate profile is calculated as the difference between the maximum release rate and the release rate achieved at the first time point after start-up (for example, at 6 hours), divided by the average release rate between the two data points.

Drug layer 30 and drug layer 40 may optionally contain surfactants and disintegrants in either or both drug layers. Exemplary of the surfactants are those having an HLB value of about 10-25, such as polyethylene glycol 400 monostearate, polyoxyethylene-4-sorbitan monolaurate, polyoxyethylene-20-sorbitan monooleate, polyoxyethylene-20-sorbitan monopalmitate, polyoxyethylene-20-monolaurate, polyoxyethylene-40 -stearate, sodium oleate and the like.

Disintegrants may be selected from starches, clays, celluloses, algins and gums and crosslinked starches, celluloses and polymers. Representative disintegrants include corn starch, potato starch, croscarmelose, crospovidone, sodium starch glycolate, Veegum HV, methylcellulose, agar, bentonite, carboxymethylcellulose, alginic acid, guar gum, low substituted hydroxypropyl cellulose and the like.

Semipermeable membrane 20 is formed to be permeable to the passage of an external fluid, such as water and biological fluids, and is substantially impermeable to the passage of drug, osmagent, osmopolymer and the like. As such, it is semipermeable as defined above.

Push layer 50 comprises an expandable layer in contacting layered arrangement with the second component drug layer 40 as illustrated in FIG. 2. Push layer 50 comprises a polymer that imbibes an aqueous or biological fluid and swells to push the drug composition through the exit of the device.

The expandable layer comprises in one embodiment a hydroactivated composition that swells in the presence of water, such as that present in gastrointestinal fluids. Conveniently, it can comprise an osmotic composition comprising an osmotic solute that exhibits an osmotic pressure gradient across the semipermeable layer against an external fluid present in the environment of use. In another embodiment, the hydro-activated layer comprises a hydrogel that imbibes and/or absorbs fluid into the layer through the outer semipermeable wall. The semipermeable wall is non-toxic. It maintains its physical and chemical integrity during operation and it is essentially free of interaction with the expandable layer.

The expandable layer in one preferred embodiment comprises a hydroactive layer comprising a hydrophilic polymer, also known as osmopolymers. The osmopolymers exhibit fluid imbibition properties. The osmopolymers are swellable, hydrophilic polymers, which osmopolymers interact with water and biological aqueous fluids and swell or expand. The osmopolymers exhibit the ability to swell in water and biological fluids and retain a significant portion of the imbibed fluid within the polymer structure during the functional lifetime of the delivery system. The osmopolymers swell or expand to a very high degree, usually exhibiting a 2 to 50 fold volume increase. The osmopolymers can be non-cross-linked or cross-linked. The swellable, hydrophilic polymers are in one embodiment lightly cross-linked, such cross-links being formed by covalent or ionic bonds or residue crystalline regions after swelling. The osmopolymers can be of plant, animal or synthetic origin.

The osmopolymers are hydrophilic polymers. Hydrophilic polymers suitable for the present purpose include poly (hydroxy-alkyl methacrylate) having a molecular weight of from 30,000 to 5,000,000; poly (vinylpyrrolidone) having a molecular weight of from 10,000 to 360,000; anionic and cationic hydrogels; polyelectrolytes complexes; poly (vinyl alcohol) having a low acetate residual, cross-linked with glyoxal, formaldehyde, or glutaraldehyde and having a degree of polymerization of from 200 to 30,000; a mixture of methyl cellulose, cross-linked agar and carboxymethyl cellulose; a mixture of hydroxypropyl methylcellulose and sodium carboxymethylcellulose; a mixture of hydroxypropyl ethylcellulose and sodium carboxymethyl cellulose, a mixture of sodium carboxymethylcellulose and methylcellulose, sodium carboxymethylcellulose; potassium carboxymethylcellulose; a water insoluble, water swellable copolymer formed from a dispersion of finely divided copolymer of maleic anhydride with styrene, ethylene, propylene, butylene or isobutylene crosslinked with from 0.001 to about 0.5 moles of saturated cross-linking agent per mole of maleic anhydride per copolymer; water swellable polymers of N-vinyl lactams; polyoxyethylene-polyoxypropylene gel; carob gum; polyacrylic gel; polyester gel; polyether gel, polyamide gel; initially dry hydrogels that imbibe and absorb water which penetrates the glassy hydrogel and lowers its glass temperature; and the like.

Representative of other osmopolymers are polymers that form hydrogels such as Carbopol™. acidic carboxypolymer, a polymer of acrylic acid cross-linked with a polyallyl sucrose, also known as carboxypolymethylene, and carboxyvinyl polymer having a molecular weight of 250,000 to 4,000,000; Cyanomer™ polyacrylamides; cross-linked water swellable indenemaleic anhydride polymers; Good-rite™ polyacrylic acid having a molecular weight of 80,000 to 200,000; Polyox™ polyethylene oxide polymer having a molecular weight of 100,000 to 5,000,000 and higher; starch graft copolymers; Aqua-Keeps™ acrylate polymer polysaccharides composed of condensed glucose units such as diester cross-linked polygluran; and the like. Representative polymers that form hydrogels are known to the prior art in U.S. Pat. No. 3,865,108; U.S. Pat. No. 4,002,173; U.S. Pat. No. 4,207,893; and in Handbook of Common Polymers, by Scott and Roff, published by the Chemical Rubber Co., Cleveland, Ohio. The amount of osmopolymer comprising a hydro-activated layer can be from about 5% to 100%.

The expandable layer in another manufacture can comprise an osmotically effective compound that comprises inorganic and organic compounds that exhibit an osmotic pressure gradient across a semipermeable wall against an external fluid. The osmotically effective compounds, as with the osmopolymers, imbibe fluid into the osmotic system, thereby making available fluid to push against the inner wall, i.e., in some embodiments, the barrier layer and/or the wall of the soft or hard capsule for pushing active agent from the dosage form. The osmotically effective compounds are known also as osmotically effective solutes, and also as osmagents. Osmotically effective solutes that can be used comprise magnesium sulfate, magnesium chloride, sodium chloride, potassium sulfate, sodium sulfate, lithium sulfate, potassium acid phosphate, mannitol, urea, inositol, magnesium succinate, tartaric acid, carbohydrates such as raffinose, sucrose, glucose, lactose, sorbitol, and mixtures thereof. The amount of osmagent in can be from about 5% to 100% of the weight of the layer. The expandable layer optionally comprises an osmopolymer and an osmagent with the total amount of osmopolymer and osmagent equal to 100%. Osmotically effective solutes are known to the prior art as described in U.S. Pat. No. 4,783,337.

Inner wall 90 further provides a lubricating function that facilitates the movement of first drug layer 30, second drug layer 40 and push layer 50 toward exit 60. Inner wall 90 may be formed from hydrophilic materials and excipients. Semipermeable membrane 20 is semipermeable, allowing gastrointestinal fluid to enter the compartment, but preventing the passage of the materials comprising the core in the compartment. The deliverable drug formulation is released from exit port 60 upon osmotic operation of the osmotic oral dosage form.

Inner wall 90 also reduces friction between the external surface of drug layer 30 and drug layer 40, and the inner surface of semipermeable membrane 20. Inner wall 90 promotes release of the drug composition from the compartment and reduces the amount of residual drug composition remaining in the compartment at the end of the delivery period, particularly when the slurry, suspension or solution of the drug composition that is being dispensed is highly viscous during the period of time in which it is being dispensed. In dosage forms with hydrophobic agents and no inner wall, it has been observed that significant residual amounts of drug may remain in the device after the period of delivery has been completed. In some instances, amounts of 20% or greater may remain in the dosage form at the end of a twenty-four hour period when tested in a release rate assay.

Inner wall 90 is formed as an inner coat of a flow-promoting agent, i.e., an agent that lowers the frictional force between the semi-permeable membrane 20 and the external surface of drug layer 40. Inner wall 90 appears to reduce the frictional forces between semi-permeable membrane 20 and the outer surface of drug layer 30 and drug layer 40, thus allowing for more complete delivery of drug from the device. Particularly in the case of active compounds having a high cost, such an improvement presents substantial economic advantages since it is not necessary to load the drug layer with an excess of drug to insure that the minimum amount of drug required will be delivered. Inner wall 90 may be formed as a coating applied over the compressed core.

Inner wall 90 typically may be 0.01 to 5 mm thick, more typically 0.5 to 2 mm thick, and it comprises a member selected from hydrogels, gelatin, low molecular weight polyethylene oxides, e.g., less than 100,000 MW, hydroxyalkylcelluloses, e.g., hydroxyethylcellulose, hydroxypropylcellulose, hydroxyisopropylcelluose, hydroxybutylcellulose and hydroxyphenylcellulose, and hydroxyalkyl alkylcelluloses, e.g., hydroxypropyl methylcellulose, and mixtures thereof. The hydroxyalkylcelluloses comprise polymers having a 9,500 to 1,250,000 number-average molecular weight. For example, hydroxypropyl celluloses having number average molecular weights of 80,000 to 850,000 are useful. The inner wall may be prepared from conventional solutions or suspensions of the aforementioned materials in aqueous solvents or inert organic solvents.

Preferred materials for the inner wall include hydroxypropyl cellulose, hydroxyethyl cellulose, hydroxypropyl methyl cellulose, povidone [poly(vinylpyrrolidone)], polyethylene glycol, and mixtures thereof.

Most preferred are mixtures of hydroxypropyl cellulose and povidone, prepared in organic solvents, particularly organic polar solvents such as lower alkanols having 1-8 carbon atoms, preferably ethanol, mixtures of hydroxyethyl cellulose and hydroxypropyl methyl cellulose prepared in aqueous solution, and mixtures of hydroxyethyl cellulose and polyethylene glycol prepared in aqueous solution. An especially preferred inner wall composition comprises a blend of hydroxyethyl cellulose having a molecular weight of approximately 90,000 and 5 wt % to 50 wt % of polyethylene glycol with a molecular weight of about 8,000. Most preferably, the inner wall comprises a mixture of hydroxypropyl cellulose and povidone prepared in ethanol.

It is preferred that inner wall 90 comprises between about 50% and about 90% hydroxypropylcellulose identified as EF having an average molecular weight of about 80,000 and between about 10% and about 50% polyvinylpyrrolidone identified as K29-32.

Conveniently, the weight of the inner wall applied to the compressed core may be correlated with the thickness of the inner wall and residual drug remaining in a dosage form in a release rate assay such as described herein. As such, during manufacturing operations, the thickness of the inner wall may be controlled by controlling the weight of the inner wall taken up in the coating operation.

When inner wall 90 is formed as a subcoat, i.e., by coating onto the tabletted composite including one or all of the first drug layer, second drug layer and push layer, the inner wall can fill in surface irregularities formed on the core by the tabletting process. The resulting smooth external surface facilitates slippage between the coated composite core and the semipermeable wall during dispensing of the drug, resulting in a lower amount of residual drug composition remaining in the device at the end of the dosing period. When inner wall 90 is fabricated of a gel-forming material, contact with water in the environment of use facilitates formation of the gel or gel-like inner coat having a viscosity that may promote and enhance slippage between semi-permeable membrane 20 and drug layer 30 and drug layer 40.

Pan coating may be conveniently used to provide the completed dosage form, except for the exit orifice. In the pan coating system, the wall-forming composition for the inner wall or the outer wall, as the case may be, is deposited by successive spraying of the appropriate wall composition onto the compressed trilayered or multilayered core comprising the drug layers, optional barrier layer and push layer, accompanied by tumbling in a rotating pan. A pan coater is used because of its availability at commercial scale. Other techniques can be used for coating the compressed core. Once coated, the wall is dried in a forced-air oven or in a temperature and humidity controlled oven to free the dosage form of solvent(s) used in the manufacturing. Drying conditions will be conventionally chosen on the basis of available equipment, ambient conditions, solvents, coatings, coating thickness, and the like.

Other coating techniques can also be employed. For example, the wall or walls of the dosage form may be formed in one technique using the air-suspension procedure. This procedure consists of suspending and tumbling the compressed core in a current of air and the semipermeable wall forming composition, until the wall is applied to the core. The air-suspension procedure is well suited for independently forming the wall of the dosage form. The air-suspension procedure is described in U.S. Pat. No. 2,799,241; in J. Am. Pharm. Assoc., Vol. 48, pp. 451-459 (1959); and, ibid., Vol. 49, pp. 82-84 (1960). The dosage form also can be coated with a Wurster® air-suspension coater using, for example, acetone with water as a cosolvent for the wall forming material. An Aeromatic® air-suspension coater can be used employing a cosolvent.

In an embodiment, the sustained release dosage form of the invention is provided with at least one exit port 60 as shown in FIG. 2. Exit port 60 cooperates with the compressed core for the uniform release of drug from the dosage form. The exit port can be provided during the manufacture of the dosage form or during drug delivery by the dosage form in a fluid environment of use.

One or more exit orifices are drilled in the drug layer end of the dosage form, and optional water soluble overcoats, which may be colored (e.g., Opadry colored coatings) or clear (e.g., Opadry Clear), may be coated on the dosage form to provide the finished dosage form.

Exit port 60 may include an orifice that is formed or formable from a substance or polymer that erodes, dissolves or is leached from the outer wall to thereby form an exit orifice. The substance or polymer may include, for example, an erodible poly(glycolic) acid or poly(lactic) acid in the semipermeable wall; a gelatinous filament; a water-removable poly(vinyl alcohol); a leachable compound, such as a fluid removable pore-former selected from the group consisting of inorganic and organic salt, oxide and carbohydrate.

An exit port, or a plurality of exit ports, can be formed by leaching a member selected from the group consisting of sorbitol, lactose, fructose, glucose, mannose, galactose, talose, sodium chloride, potassium chloride, sodium citrate and mannitol to provide a uniform-release dimensioned pore-exit orifice.

The exit can have any shape, such as round, triangular, square, elliptical and the like for the uniform metered dose release of a drug from the dosage form.

The sustained release dosage form can be constructed with one or more exits in spaced-apart relation or one or more surfaces of the sustained release dosage form.

Drilling, including mechanical and laser drilling, through the semipermeable wall can be used to form the exit orifice. Such exits and equipment for forming such exits are disclosed in U.S. Pat. Nos. 3,916,899, by Theeuwes and Higuchi and in U.S. Pat. No. 4,088,864, by Theeuwes, et al. It is presently preferred to utilize two exit ports of equal diameter. In a preferred embodiment, exit port 60 penetrates through subcoat 90, if present, to drug layer 30.

In another embodiment, the drug and other ingredients comprising a therapeutic composition or comprising the drug layer facing the exit are blended, or they are blended then pressed, into a solid layer. The drug and other ingredients can be blended with a solvent and formed into a solid or semisolid formed by conventional methods such as ball-milling, calendering, stirring or roll-milling and then pressed into a selected shape. The layer possesses dimensions that correspond to the internal dimensions of the area the layer is to occupy in the dosage form. The bilayer possesses dimensions corresponding to the internal lumen of the dosage form. Next, a hydrogel “push-layer” is placed in contact with the drug layer. The layering of the drug layer and the hydrogel push layer can be fabricated by conventional press-layering techniques. Finally, the two-layer compartment forming members are surrounded and coated with an outer wall. A passageway is laser drilled or mechanically drilled through the wall to contact the drug layer, with the dosage form optically oriented automatically by the laser equipment for forming the passageway on the pre-selected drug surface.

In another embodiment, the dosage form is manufactured by the wet granulation technique. In the wet granulation technique, the drug and the ingredients comprising the first layer are blended using an organic or inorganic solvent, such as isopropyl alcohol:methanol 80:20 (v:v) as the granulation fluid. Other granulating fluid, such as water, isopropyl alcohol, or denatured alcohol 100% can be used for this purpose. The ingredients forming the drug layer are individually passed through a 40-mesh screen, then thoroughly blended in a mixer. Next, other ingredients comprising the drug layer are dissolved in a portion of the granulation fluid, such as the cosolvent described above. Then, the latter prepared wet blend is slowly added to the drug blend with continual mixing in the blender. The granulating fluid is added until a wet blend mass is produced, which wet mass is then forced through a 20-mesh screen onto oven trays. The blend is dried for 18 to 24 hours at 25° C. to 40° C. The dry granules are then screened with a 16-mesh screen. Next, a lubricant is passed through a 60-mesh screen and added to the dry screened granule blend. The granulation is put into milling jars and mixed on a jar mill for 1 to 5 minutes. The drug layer and push-layer compositions are pressed into a layered tablet, for example, on a Manesty® layer press.

Another manufacturing process that can be used for providing the drug and hydrogel composition comprises blending their powdered ingredients in a fluid-bed granulator. After the powdered ingredients are dry blended in the granulator, a granulating fluid, for example, poly(vinylpyrrolidone) in a solvent, such as in water, is sprayed onto the respective powders. The coated powders are then dried in the granulator. This process coats the dry ingredients present therein while spraying the granulating fluid. After the granules are dried, a lubricant, such as stearic acid or magnesium stearate, is blended as above into the mixture. The granules are then pressed in the manner described above. In another embodiment, when the fluid-bed granulating process is used to manufacture the hydrogel layer, the antioxidant present in the polyalkylene oxide can be removed during the processing step. If antioxidant is desired, it can be added to the hydrogel formulation; this can be accomplished during the fluid-bed granulation described above.

Dosage forms according to the invention may be manufactured in another embodiment by mixing the drug with composition-forming ingredients and pressing the composition into a solid layer possessing dimensions that correspond to the internal dimensions of the compartment space adjacent to a passageway. In another embodiment, the drug and other drug composition forming ingredients and a solvent are mixed into a solid, or semi-solid, by conventional methods such as ball-milling, calendering, stirring or roll-milling, and then pressed into a preselected, layer-forming shape.

In the embodiment presented above, the composition or a layer of the composition comprising a hydrogel osmopolymer and an optional osmagent is placed in contact with the layer comprising the drug, and the two layers are surrounded with a semipermeable membrane. The layering of the drug composition and the hydrogel push-layer and optional osmagent composition can be accomplished by using a conventional two-layer tablet press technique. The wall can be deposited through the molding, spraying, or dipping of pressed shapes with semi-permeable membrane forming materials. Another technique that can be used for applying the semi-permeable membrane is the air-suspension coating procedure. This procedure consists in suspending and tumbling the two layers in a current of air until the semi-permeable membrane forming composition surrounds the layers. Alternatively, the semi-permeable membrane may be formed through a pan coating process, wherein the pressed shapes are tumbled in a pan while the semi-permeable membrane forming composition is sprayed onto said shapes. Manufacturing procedures are described in Modern Plastics Encyclopedia, Vol. 46, pp. 62-70 (1969); and in Pharmaceutical Sciences, by Remington, 14th Ed., pp. 1626-1648 (1970), published by Mack Publishing Co., Easton, Pa. The dosage form can be manufactured by following the teaching in U.S. Pat. Nos. 4,327,725; 4,612,008; 4,783,337; 4,863,456; and 4,902,514.

Exemplary solvents suitable for manufacturing the semipermeable membrane, the composition layers and the dosage form generally include inert inorganic and organic solvents that do not adversely harm the materials, the wall, the layer, the composition and the drug wall. Any flux enhancers in the wall composition are first dissolved in the solvent under stirring with or without the aid of heat. Such solvents may be aqueous, organic, or mixtures thereof. After complete dissolution of the flux enhancers, the cellulosic component, cellulose acetate, for example, of the wall-forming material is added and stirring is continued until both components are in solution. The membrane may be applied onto the core by using a Wurster coater, pan coater or any other coating equipment. Alternatively a blend of the coating materials may also be directly compressed onto the said core. A desirable thickness of the semi-permeable membrane is approximately 4 to 6 mils.

The resulting membranes of the coated cores may be drilled using a Servo drill press fitted with a 25 mil bit and dried at 45° C./45% Rh for 72 hours in a hot pack humidity oven followed by 4 hours drying at 45° C. to remove excess moisture.

Exit ports of the coated systems are typically drilled through the rate controlling membrane prior to drying to provide a means for residual solvents to be removed. Drying processes on the drilled systems are typically conducted in ovens thermostated at 45° C. in 45% relative humidity air, typically for a period of approximately 72 hours. The moisture of the humid air helps to plasticize the membrane such that diffusion of the residual coating solvents from the dosage form through the membrane can be accelerated and the overall drying process time shortened. A final drying step can be included to remove residual water introduced by humidification by treating the batch without humidity for a few hours at 45° C. The duration of the drying process is typically 1 to 10 days with a preferred duration of typically 1-3 days.

While there has been described and pointed out features and advantages of the invention, as applied to present embodiments, those skilled in the medical art will appreciate that various modifications, changes, additions, and omissions in the method described in the specification can be made without departing from the spirit of the invention.

### EXAMPLES

The following examples are meant to be illustrative of the claimed invention and not limiting in any manner.

### Example 1

The fundamental transport and mechanical properties of membranes comprised of cellulose acetate blended with acrylate copolymer were characterized in a series of experiments. First, a latex of ethyl acrylate methyl methacrylate (EMM) commercially available as Eudragit® NE30 was dried in a forced air oven to remove the water fraction of the latex. The EMM had a monomer ratio of approximately 2 parts ethyl acrylate and 1 part methyl methacrylate. Molecular weight of the polymer was approximately 800,000 grams per mole. 75 grams of the resulting dried solids were then recovered and dissolved with stirring in 3000 grams of acetone. Next, 75 grams of cellulose acetate 39.8 (CA 398) was added to the blend and stirred to dissolution. The cellulose acetate had an average acetyl content of 39.8 weight percent, a falling ball viscosity of 10 seconds, and an average molecular weight of approximately 40,000 grams per mole. The resulting composition formed the coating solution.

The coating solution was then spray coated to form planar membrane samples in a pharmaceutical coating pan. A 1-kilogram charge of ⅜-inch lactose tablets and about a dozen flat-faced 1-inch diameter Delrin discs were then charged into a Vector pan Hi-Coater fitted with a 12-inch pan. The coating solution was spray coated through an air atomizing nozzle onto the Delrin discs that were suspended in a tumbling bed of the lactose tablets in a current of warm drying air using an inlet temperature of about 40-42° C. and an outlet temperature of about 27° C. to remove the acetone. The coating process continued until the membrane coating was accumulated on the discs. The coated discs were then removed from the coater and air dried overnight remove residual acetone. The resulting membrane composition was 50 wt % CA 398 and 50 wt % EMM.

The membrane samples were peeled from the discs to yield the free membranes for further testing.

Osmotic permeability of the resulting membrane was measured in a two-compartment Franz cell. The membrane was supported from flexing by a 50 mesh stainless steel screen which screen and membrane were sandwiched between the two compartments. One compartment was filled with a saturated solution with excess solid sodium chloride and the other compartment was filled with de-ionized water. The cell was maintained at a constant temperature of 37° C. and a graduated pipet was attached to the compartment of the salt solution for measuring volumetric flow. As water was drawn across the membrane by osmosis, the volumetric flow of water was monitored on the pipet as a function of time. When equilibrium flow is reached, the osmotic permeability, K, was then calculated according to Equation 1:
K=[(dv/dt)(h)]/(Π)(A) Eq 1

Where dv/dt=volumetric flow per unit time (cm³/min)

h=membrane thickness (mils)

Π=osmotic pressure (atmospheres)

A=membrane area (cm²)

The value of osmotic pressure of saturated sodium chloride solution for use in this equation was independently measured with a vapor pressure osmometer to yield a value of 387 atmospheres. The average value of osmotic permeability was determined from measuring permeability values of least 3 membranes.

This experiment was then repeated according to the same procedures except that the water from the latex was not pre-dried prior to making the coating solution. The resulting solvent system for the coating solution was therefore 94 wt % acetone and 6 wt % water. The 6 wt % water was therefore introduced by the water originally present within the latex.

This experiment was again repeated but with 100% CA 398 coated from 100% acetone.

Finally, this experiment was again repeated but with 50% CA 398 and 50% polyvinyl acetate sprayed from 100% acetone. The polyvinyl acetate was commercially available as Sentry Plus Resin GB-50 from Dow Chemical. It has a molecular weight in the range of 45,000 to 54,000 grams it was selected as a candidate to blend with cellulose acetate as both polymers have a high fraction of like acetyl groups and are of similar molecular weight.

A summary of the solvent compositions and permeability values are shown in Table 1. The osmotic permeability values of the membranes blended with 50 wt % ethyl acrylate methyl methacrylate are approximately one third the permeability value of 100% cellulose acetate. Similarly, the permeability value for the membrane blended with 50 wt % polyvinyl acetate (PVAc) are approximately one third the permeability of 100% cellulose acetate. Within experimental error, the permeability values of the 50:50 CA:NE blends coated from a solvent of pure acetone were comparable to the permeability of this membrane coated from 94/6 acetone/water wt/wt.

TABLE 1
Permeability of Cellulose Acetate 398 Neat, and Blended
with Ethyl Acrylate Methyl Methacrylate or Polyvinyl Acetate.

| | Solvent System | Permeability exp 5 |
|---|---|---|
| Membrane (wt %) | (acetone/water) | (cm mil/atm hr) ± sd |
| 100 CA 398-10 | 100/0 | 5.9 ± 0.2 |
| 50/50 CA 398/NE | 94/6 | 2.0 ± 0.1 |
| 50/50 CA 398/NE | 100/0 | 1.6 ± 0.3 |
| 50/50 CA 398/PVAc | 100/0 | 1.7 ± 0.4 |

### Example 2

Mechanical properties of the membrane compositions from Example 1 were characterized. Prior to testing, samples were punched with a dog bone shaped die, dried overnight in forced air at 40° C., then re-humidified to constant weight at 52% relative humidity until they reached equilibrium weight. The dog bone die was 0.35 inch long, 0.25 inch wide with a 0.125 inch wide neck. The oven drying was done to remove residual acetone coating solvent. The samples were treated to the humidity condition to return the moisture levels in the samples to that approximating equilibrium moisture at ambient humidity.

Half of the samples were immersed in de-ionized water at room temperature for at least 4 hours prior to tensile testing while the other half remained dry. Elastic modulus and elongation at break were then measured on both sets of samples using an Instron Corporation Series IX Automated Materials Testing System. Five samples of each membrane type were pulled with a crosshead speed of 10 mm per minute. Elastic modulus represents the stiffness of the membrane and elongation a break represents its ability to stretch prior to breaking. Table 2 illustrates the results of these tests.

TABLE 2
Tensile Properties of Dry and Wet Membranes, of Cellulose
Acetate 398-10 Neat or Blended with 50% of Ethyl Acrylate
Methyl Methacrylate or with 50% Polyvinyl Acetate.

| | | | Elongation |
|---|---|---|---|
| Membrane | Solvent System | Elastic Modulus | at Break |
| (wt %) | (acetone/water) | (psi) exp 4 ± sd | (%) ± sd |
| | | Dry | |
| 100 CA 398-10 | 100/0 | 13.1 ± 2.3 | 19 ± 7 |
| 50/50 CA 398/NE | 94/6 | 4.7 ± 0.5 | 43 ± 5 |
| 50/50 CA 398/NE | 100/0 | 3.7 ± 1.5 | 44 ± 5 |
| 50/50 CA 398/PVAc | 100/0 | 8.8 ± 2.5 | 10 ± 4 |
| | | Wet | |
| 100 CA 398-10 | 100/0 | 8.9 ± 2.1 | 38 ± 16 |
| 50/50 CA 398/NE | 94/6 | 2.9 ± 0.7 | 50 ± 7 |
| 50/50 CA 398/NE | 100/0 | 1.6 ± 0.9 | 73 ± 13 |
| 50/50 CA 398/PVAc | 100/0 | 8.0 ± 1.4 | 16 ± 5 |

Referring to the results of membranes tested in the dry state, the neat cellulose acetate 398-10 had a higher modulus of elasticity than the blended membranes, however, the CA 398/NE blends had higher values for elongation at break than the neat CA 398-10. Referring to the results in the wet (hydrated) state, although the CA/PVAc had an elastic modulus comparable to neat CA 398-10, the CA 398/PVAc blend was brittle, as evidenced by the much lower elongation at break. The CA/PVAc blend likewise had a low elongation at break in the dry state compared to the neat CA 398-10 and the 50:50 CA 398-10/NE blends.

These results illustrate the difficulty of predicting a priori a membrane composition that would have good functional properties. Although the polyvinyl acetate has the acetyl functional groups common to cellulose acetate and has a molecular weight similar to cellulose acetate 398-10, the 50:50 CA 398-10:PVAc blend tends to be brittle. By contrast, the 50:50 blend of CA 398-10:NE has good mechanical properties despite the lack of common functional groups and markedly dissimilar molecular weights.

### Example 3

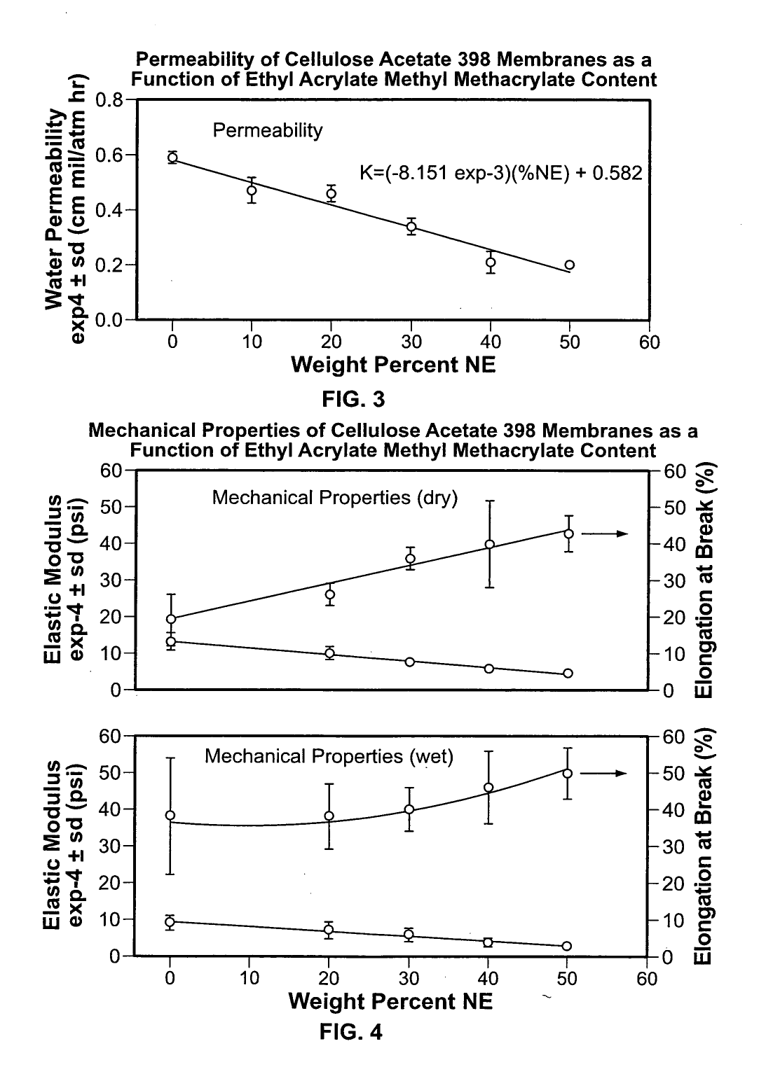

Spray formed membranes comprising cellulose acetate 398-10 and ethyl acrylate methyl methacrylate copolymer (Eudragit NE) with various ratios of the polymers were fabricated according to the procedures described in Example 1. In each case, the coating solution was prepared by dissolving the latex as received from the manufacturer including the water fraction in the acetone solvent. Then the cellulose acetate was dissolved in the resulting acetone/water blend. Permeability values were measured as described in Example 1. FIG. 3 summarizes the results. The reduction in permeability follows a linear function of the weight fraction of the ethyl acrylate methyl methacryate as described by the equation in FIG. 3. The permeability values of these membranes are in useful ranges for osmotic delivery systems.

### Example 4

Spray formed membranes comprising cellulose acetate 398-10 and ethyl acrylate methyl methacrylate copolymer (Eudragit NE) with various ratios of the polymers were fabricated according to the procedures described in Example 1. In each case, the coating solution was prepared by dissolving the latex as received from the manufacturer including the water fraction was dissolved in the acetone. Next, the cellulose acetate was dissolved in the resulting acetone/water blend. Mechanical properties of the dry and wet (hydrated) membranes were measured as described in Example 2. FIG. 4 summarizes the results.

Elongation at break increases as the fraction of acrylate copolymer increases in both dry and wet membranes. Elastic modulus decreases linearly as the fraction of acrylate copolymer increases in both wet and dry membranes. The tensile values of these membranes are in the useful ranges for osmotic delivery systems.

### Example 5

Performance of the cellulose acetate acrylate copolymer membrane was evaluated on an osmotic dosage form. Osmotic tablets of the anti-depressant drug, reboxetine methane sulfonate, were fabricated using conventional fluidized bed granulation, tri-layer tablet compression, and pan spray coating processes. Tablets were compressed with longitudinally compressed tooling with three layers that consist of maltodextrin-based drug layer, an ethylcellulose barrier layer, and a polyethylene oxide-based push layer to produce a core that was 4.8 mm diameter and 13 mm long. 145 mg of the drug layer composition was first lightly compressed. This drug layer composition comprised 9 weight percent of reboxetine methane sulfonate 85.5 weight percent of maltodextrin M100 osmotic agent, 2 weight percent of stearic acid tablet lubricant, and 3.5 weight percent water. The drug layer contained 10.0 mg as equivalent to reboxetine free base. Next, 30 mg of barrier layer was lightly compressed onto the drug layer composition. The barrier layer comprised 99 weight percent of ethyl cellulose standard premium viscosity 100 cps barrier polymer, and 1 weight percent stearic acid tablet lubricant. Finally, 97 mg of push layer composition was compressed onto the barrier layer with a final compression force sufficient to laminate a cohesive bond between the three compressed layers. This formed the osmotic tablet. Next, 25 mg of subcoat film was applied onto the resulting tri-layer tablets by spray coating in a coating pan a film composition from a spray coating solution comprising 6 weight percent solids of film composition dissolved in 100 weight percent of ethyl alcohol. The composition of the subcoat film was 70 weight percent of hydroxypropyl cellulose EF and 30 weight percent polyvinyl pyrrolidone K2932. The resulting subcoated trilayer osmotic tablets weighing a total of 297 mg were then used in subsequent coating studies to evaluate low permeability membranes.

Three coating trials were next conducted using these trilayer osmotic reference cores. The experimental control comprising the prior art membrane of 100% cellulose acetate was first coated. 200 grams of cellulose acetate 398-10 were dissolved in 3800 grams of acetone with stirring at room temperature. Approximately 600 of the osmotic reference cores were mixed with 820 grams of 9/32 inch diameter lactose tablets and the mixture of tablets was charged into a Vector LDCS pan coater fitted with a 12 inch diameter pan. The coating solution was stirred continuously during the application process and was sprayed at a rate of 22-24 ml per minute onto the charge of tablets while tumbling in a current of warm drying air. The inlet temperature was maintained at 43-41° C. and the outlet temperature was maintained at 23-26° C. The coating proceeded for 135 minutes until a coating weight of 49 mg and coating thickness of 7.1 mil was accumulated on the reference cores. The batch of osmotic reference cores was then separated from the lactose filler tablets. The membrane-coated systems were then drilled with two exit ports on the dome of the drug layer end of the tablet using a drill press fitted with a 15-mil bit. A portion of the drilled batch was refrigerated, another small portion of the batch was frozen for separate studies. Five systems from the batch were placed in a drying oven maintained at 45° C. and 45% relative humidity.

After drying for ten days, the 5 systems were tested for release of drug. The systems were suspended in stainless less baskets and released into receptors containing 45 ml of de-ionized water thermostated to 37° C. using U.S. Pharmacopeia 26/National Formulary 21 Apparatus 7 for two hours. The test systems were then transferred to fresh receptors and allowed to release for two more hours. This process was repeated to accumulate 12 intervals representing 24 hours of delivery performance. The receptor solutions then filtered through a 0.8 micron filter and drug concentration was analyzed by ultraviolet spectroscopy using a wavelength of 273.8 nm. Release rate of drug was plotted as a function of time. The data are shown in the upper frame of FIG. 3. The system delivered the drug at a steady state rate of about 0.7 mg per hour during the steady state duration between 2 hours to 14 hours. The time to deliver 90% of the claimed dose was 16.5 hours.

The second batch of reference osmotic tablets was then coated. First, 75 grams of Eudragit NE30 as an aqueous latex was added with stirring to 2797.5 grams of acetone. The Eudragit NE30 is supplied by Rohm America Incorporated, Somerset, N.J. The 75-gram sample of latex contained 22.5 grams of ethyl acrylate methyl methacrylate 2:1 copolymer and 52.5 grams of water. Then, 127.5 grams of cellulose acetate 398-10 was added and stirred to complete dissolution. Approximately 600 of the osmotic reference cores from Example 5 were mixed with 820 grams of 9/32 inch diameter lactose tablets and the mixture of tablets was charged into a Vector LDCS pan coater fitted with a 12 inch diameter pan. The coating solution was then applied using the parameters described in the first batch. The coating process continued for 71 minutes until 29 mg and a thickness of 4.5 mils was deposited on the osmotic reference tablets. The composition of the resulting membrane was 85 weight percent cellulose acetate 398-10 and 15 weight percent Eudragit NE. The resulting membrane coated systems were then drilled as described in batch one. A portion of the drilled batch was refrigerated, another small portion of the batch was frozen for separate studies. Five systems from the batch were placed in a drying oven maintained at 45° C. and 45% relative humidity.

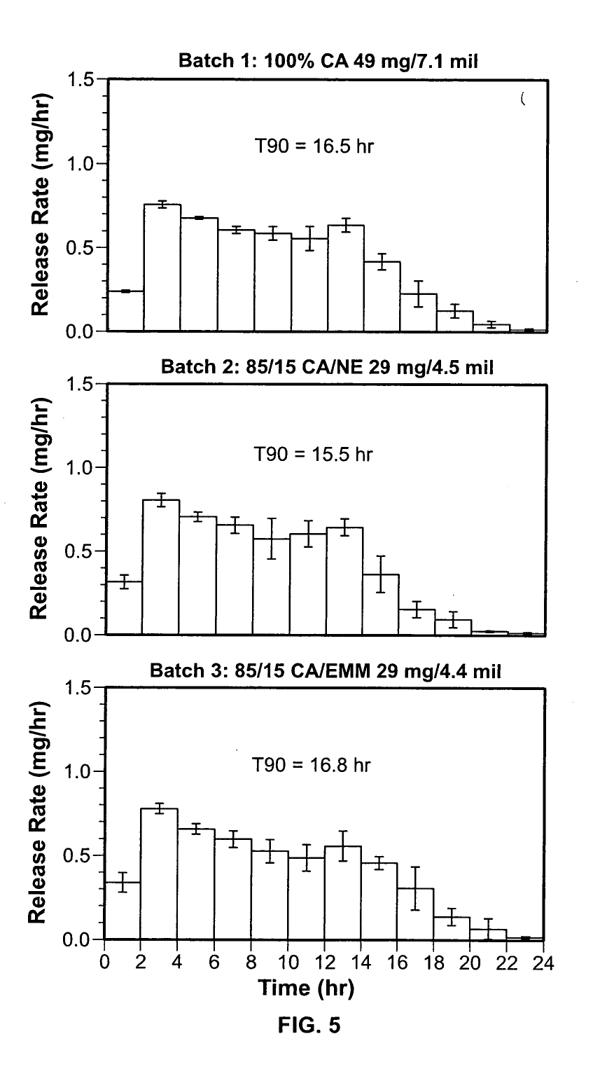

The resulting drilled and dried membrane coated systems were tested for release of drug according to the procedures described for the testing of drug release from systems of batch one. The data are shown in the middle frame of FIG. 5. The system delivered the drug at a steady state rate in the range of about 0.7 mg per hour during the steady state duration between 2 hours to 14 hours. The time to deliver 90% of the claimed dose was 15.5 hours.

Finally, a third batch of reference osmotic tablets was coated. The composition of the coating solution applied to this batch was equivalent to batch two except the ethyl acrylate methyl methacrylate 2:1 copolymer was Kollicoat EMM. This latex is supplied by BASF Corporation, Mount Olive, N.J. The membrane coating process, drilling, and drying, and release rate testing were equivalent to those used to manufacture batch two. A coating weight of 29 mg and coating thickness of 4.4 mils was applied of the final membrane composition comprising 85 weight percent cellulose acetate 398-10 and 15 weight percent of Kollicoat EMM. The processing time to reach this coating weight was 66 minutes.

The delivery pattern of batch three is depicted in the bottom frame of FIG. 5. The system delivered the drug approximately at a rate of 0.7 mg per hour for 14 hours. The time to deliver 90% of claimed dose was 16.8 hours.

While the delivery patterns of batches one, two and, three are comparable and time to coat the membranes in the coating process was different. The cellulose acetate acrylate blended membranes of the present invention were applied in approximately half the manufacturing process time compared to the 100% cellulose acetate membrane of the prior art which reduction in time substantially increases the capacity to manufacture products while reducing the amount of coating materials needed to be applied.

### Example 6

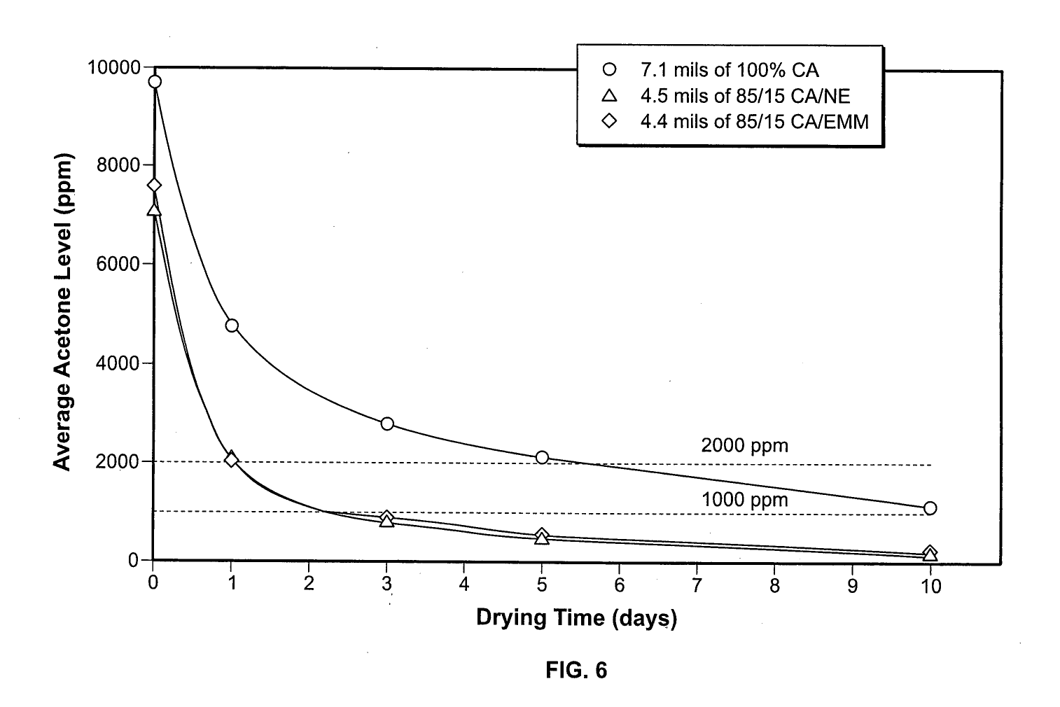

The time to remove residual coating solvent to acceptable levels of the prior art membrane was compared to the time to dry the membranes of the present invention. In process samples were taken from batches 1, 2, and 3 of example 5 after the application of the membranes and drilling the delivery ports. The osmotic delivery systems were dried in a 45° C. oven with 45 percent relative humidity and six systems of each membrane composition were sampled at 0, 1, 3, 5 and 10 days. Residual coating solvent within the dosage forms as a function of drying time was measured by high pressure liquid chromatography. The time zero sample was represented by the samples that had initially been frozen. The results of this drying study are shown in FIG. 6.

Acceptable level of residual acetone is 1000 parts per million. The membrane of the prior art represented by the coating of batch 1 with 7.1 mils of 100% cellulose acetate 398 required at least 10 days of drying to reach the acceptable level. The membranes of the present invention, represented by 4.5 mils of 85/15 cellulose acetate 398/Eudragit NE or 4.4 mils of 85/15 cellulose acetate 398/Kollicoat EMM reached acceptable levels by 3 days. The membranes of the present invention reduce drying time and therefore increase production rate substantially.

What is claimed is:

1. An osmotic dosage form comprising:
an osmotic core;
a semi-permeable membrane that surrounds the osmotic core and comprises a blend of a cellulose acetate polymer and an acrylate copolymer; and
an exit formed through the semi-permeable membrane.

2. The osmotic dosage form of claim 1, wherein the blend comprises the cellulose acetate polymer in an amount equal to or greater than about 50 wt % based on a total dry weight of the blend.

3. The osmotic dosage form of claim 2, wherein the blend comprises the cellulose acetate polymer in an amount equal to or greater than about 60 wt % based on a total dry weight of the blend.

4. The osmotic dosage form of claim 3, wherein the blend comprises the cellulose acetate polymer in an amount equal to or greater than about 70 wt %, based on a total dry weight of the blend.

5. The osmotic dosage form of claim 1, wherein the blend comprises acrylate copolymer in an amount ranging from about 0.01 wt % to about 50 wt %, based on a total dry weight of the blend.

6. The osmotic dosage form of claim 5, wherein the blend comprises acrylate copolymer in an amount ranging from about 10 wt % to about about 30 wt %, based on a total dry weight of the blend.

7. The osmotic dosage form of claim 6, wherein the blend comprises acrylate copolymer in an amount ranging from about 15 wt % to about 25 wt %, based on a total dry weight of the blend.

8. The osmotic dosage form of claim 1, wherein the blend further comprises a flux enhancer.

9. The osmotic dosage form of claim 8, wherein the amount of flux enhancer ranges from about 0.01 wt % to about 25 wt %, based on a total dry weight of the blend.

10. The osmotic dosage form of claim 9, wherein the amount of flux enhancer ranges from about 5 wt % to about 20 wt %, based on a total dry weight of the blend.

11. The osmotic dosage form of claim 10, wherein the amount of flux enhancer ranges from about 10 wt % to about 15 wt %, based on a total dry weight of the blend.

12. A method comprising:
administering the osmotic dosage form of claim 1 to a patient.

13. A method comprising:
providing an osmotic core;
coating the osmotic core with a semi-permeable membrane that comprises a blend of a cellulose acetate polymer and an acrylate copolymer;
forming an exit through the semi-permeable membrane; and
administering the coated osmotic core to a patient.

14. The method of claim 13, wherein the blend comprises the cellulose acetate polymer in an amount equal to or greater than about 50 wt % based on a total dry weight of the blend.

15. The method of claim 14, wherein the blend comprises the cellulose acetate polymer in an amount equal to or greater than about 60 wt % based on a total dry weight of the blend.

16. The method of claim 15, wherein the blend comprises the cellulose acetate polymer in an amount equal to or greater than about 70 wt %, based on a total dry weight of the blend.

17. The method of claim 13, wherein the blend comprises acrylate copolymer in an amount ranging from about 0.01 wt % to about 50 wt %, based on a total dry weight of the blend

18. The method of claim 17, wherein the blend comprises acrylate copolymer in an amount ranging from about 10 wt % to about about 30 wt %, based on a total dry weight of the blend

19. The method of claim 18, wherein the blend comprises acrylate copolymer in an amount ranging from about 15 wt % to about 25 wt %, based on a total dry weight of the blend.

20. The method of claim 13, wherein the blend further comprises a flux enhancer.

21. The method of claim 20, wherein the amount of flux enhancer ranges from about 0.01 wt % to about 25 wt %, based on a total dry weight of the blend.

22. The method of claim 21, wherein the amount of flux enhancer ranges from about 5 wt % to about 20 wt %, based on a total dry weight of the blend.

23. The method of claim 22, wherein the amount of flux enhancer ranges from about 10 wt % to about 15 wt %, based on a total dry weight of the blend.
